(19) United States
(12) Patent Application Publication
(10) Pub. No.: US 2003/0209242 A1
(43) Pub. Date: Nov. 13, 2003

(54) Apparatuses and methods for outputting data from a sedation and analgesia system

(75) Inventor: Randall S. Hickle, Lubbock, TX (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Scott Laboratories, Inc.

(21) Appl. No.: 10/369,195

(22) Filed: Feb. 20, 2003

Related U.S. Application Data
(60) Provisional application No. 60/357,635, filed on Feb. 20, 2002.

(51) Int. Cl. A61M 15/00

(52) U.S. Cl. 128/203.12

(57) ABSTRACT

The present invention comprises a printer system integral with a sedation and analgesia system having the capability to display data at spaced intervals, such as, for example, every five minutes, where the data in the previous five minutes is averaged in order to minimize the spurious impact of artifacts on the printed record. The present invention further comprises a printer system integral with a sedation and analgesia system that, upon request, prints a detailed, real time graphical display of critical parameters regarding a patient's physiology. The printer may be used with a brief memory from which an operator may request information that may have already exited an associated visual output device.

### CROSS REFERENCE TO RELATED APPLICATIONS

This application claims priority under 35 U.S.C. §119(e) from U.S. Provisional Patent Application Serial No. 60/357,635, filed Feb. 20, 2002 and incorporated herein by reference.

### FIELD OF THE INVENTION

The present invention relates in general to printers and, more particularly, to printers associated with sedation and analgesia systems.

### BACKGROUND OF THE INVENTION

In response to, among other things, market conditions and popularity amongst cost-conscious patients, out-of-hospital procedures continue to experience rapid growth. For various reasons, clinicians such as, for example, in office, ambulatory center, dental, non-hospital and hospital settings sometimes administer or supervise the delivery of sedation and analgesia without the services of trained anesthesia providers. This development has led the American Society of Anesthesiologists to issue guidelines for the delivery of sedation and analgesia by non-anesthesiologists. Because the non-hospital setting is in general not as well equipped and staffed as hospitals, malfunctions and complications (such as unintended over-medication leading to loss of consciousness and airway reflexes) may lead to severe outcomes.

A sedation and analgesia system is described in commonly assigned and co-pending U.S. patent application Ser. No. 09/324,759, filed Jun. 3, 1999. This system safely provides patients undergoing painful, uncomfortable or otherwise frightening (anxiety inspiring) medical or surgical procedures with sedative, analgesic, and/or amnestic drugs in a way that reduces the risk of overmedication, in both non-hospital and hospital settings.

In accordance with quality assurance practices that comprise current standards of care, and to comply with the Joint Commission Accreditation of Hospitals Organization (JCAHO) standards, many systems have been developed to print pertinent information relating to drug administration and patient physiology during a medical procedure. Many medical monitors produce frequent and significant artifactual data. When patient records are manually maintained by trained healthcare providers, this artifactual data is intelligently filtered, and thus, does not distort the medical record. However, many devices employed to automate the creation of a patient's medical record utilize print algorithms that may misrepresent the patient's true condition. Because the patient record is a critical medico-legal document, unsophisticated automated record keeping devices have not generally been well accepted by clinicians due to the possibility that machine error, software error, patient monitor error, or other error is recorded though not reflective of true patient condition. This concern that artifacts may be taken as fact has led many clinicians to discourage, or to simply abandon, the use of such devices.

Other medical devices have been developed that output data via a user interface such as, for example, a computer monitor or LCD screen. These devices allow a physician or nurse to see data as it occurs in real time in order to make informed and educated decisions regarding patient care. Many of these devices lack a means of capturing information displayed on the monitor and require the displayed data to be quickly analyzed before it is continuously replaced with new incoming data. During medical procedures, a clinician may be alerted by others to the display of a potential problem on the monitor, only to view the monitor after the display indicating the potential anomaly has been overwritten by newer data. Unfortunately, once the display indicating a potential anomalous event has been overwritten, the clinician may be unable to evaluate the event in order to determine its etiology and possible significance.

The concerns regarding printers that indiscriminately record all data, including artifacts, make it difficult for hospitals to comply with JCAHO standards. These standards include, for example, recording test results relevant to the management of the patient's condition, all operative and other invasive procedures performed, progress notes made by the medical staff and other authorized individuals, clinical observations, the patient's response to care, every medication ordered or prescribed for an inpatient, every medication dispensed to an ambulatory patient or an inpatient on discharge, and every dose of medication administered. If the clinician has opted not to use a printer for the aforementioned reasons, this information will have to be scribed by a nurse or attendant during a procedure, even if the attendant's time might be better allocated to direct patient care activities. The requirement that an assistant scribe the information adds a further task to be performed by the medical or surgical team resulting in an overly multi-tasked team or the requirement for additional personnel. An overly tasked team may be prone to errors resulting in irreparable damage, and the addition of personnel raises the cost of medical care. Furthermore, manual transcription may be prone to human error, especially if critical numbers reflecting patient status are inadvertently transposed or incorrectly entered.

Other devices provide data printouts, but may not give a clinician enough information with which to make an informed decision as to the significance of the data printout. These devices may print out information related, for example, to an ECG illustrating an anomalous patient episode. From this information alone, it may be difficult for the clinician to determine the importance and/or clinical relevance of the event, and whether to take potentially costly steps to remedy the possible problem, or disregard a potentially life threatening event as an anomalous patient episode. Although these devices may comply, for example, with regulations for office-based anesthesia, they may not fully meet the needs of the patient and clinician.

### SUMMARY OF THE INVENTION

The present invention comprises a printer system integral with a sedation and analgesia system having the capability to display data at spaced intervals, such as, for example, every five minutes, where the data in the previous five minutes is averaged in order to minimize the spurious impact of artifacts on the printed record. By providing a truer overall picture of the patient's condition, this printer system facilitates acceptance and use of the printer compared to machines that indiscriminately record artifacts if the latter happen to coincide with the printing interval. The printer system allows the automated entry of data integral with a sedation and analgesia system in fields of a user interface selected as important in complying with JCAHO standards in order to free up personnel to focus on the actual procedure. The user interface for the printer system has means for efficiently and conveniently entering user input into the resulting printout to make notations that document or provide additional information to clarify patient conditions, automated recordation of erroneous data, manual administration of drugs, or for any other suitable reason.

The present invention further comprises a printer system integral with a sedation and analgesia system that, upon request, prints a detailed, real time graphical display of critical parameters regarding a patient's physiology. The printer may be used with a brief memory, such as, for example, 15 seconds, in which an operator may request information that has exited the visual output device, for example, in the last 15 seconds. After such a request is made, the printer system provides the requester with a printout that contains several critical monitored patient parameters in order to accurately assess the significance of illustrated data. The printer may be turned on or off depending on the needs of the physician.

### BRIEF DESCRIPTION OF THE DRAWINGS

FIG. 1 is an overall conceptual schematic block diagram of the apparatus in accordance with the present invention;

FIG. 2 is a more detailed schematic block diagram of the printer system in accordance with the present invention;

FIG. 3 is a view of an example of an interface prompt in accordance with the present invention;

FIG. 4 is a view of an example of a second interface prompt in accordance with the present invention;

FIG. 5 is a view of an example of a STAT printout prompt in accordance with the present invention;

FIG. 6 is a view of an example of a graph printout in accordance with the present invention;

FIG. 7 is a view of an example of a data printout in accordance with the present invention; and

FIG. 8 is a flow chart illustrating a method in accordance with the present invention.

### DETAILED DESCRIPTION OF THE INVENTION

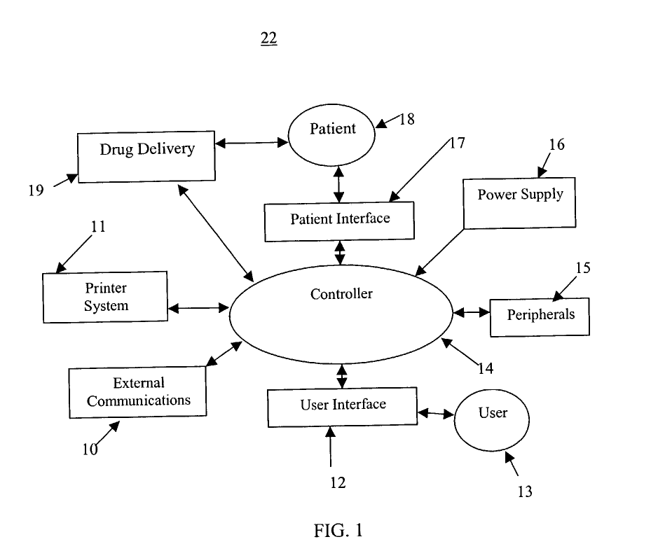

FIG. 1 illustrates a flow chart depicting one embodiment of the present invention comprising a sedation and analgesia system 22 having user interface 12, such as that described in commonly assigned and co-pending U.S. patent application Ser. No. 10/285,689 filed Nov. 1, 2002, controller 14, peripherals 15, power supply 16, external communications 10, printer system 11, patient interface 17, and drug delivery 19 where sedation and analgesia system 22 is operated by user 13 in order to provide sedation and/or analgesia to patient 18. An example of sedation and analgesia system 22 is described in commonly assigned and co-pending U.S. patent application Ser. No. 09/324,759, filed Jun. 3, 1999 and incorporated herein by reference.

The sedation and analgesia system of application Ser. No. 09/324,759 includes a patient health monitor device adapted so as to be coupled to a patient and generate a signal reflecting at least one physiological condition of the patient, a drug delivery controller supplying one or more drugs to the patient, a memory device storing a safety data set reflecting safe and undesirable parameters of at least one monitored patient physiological condition, and an electronic controller interconnected between the patient health monitor, the drug delivery controller, and the memory device storing the safety data set; wherein said electronic controller receives said signals and in response manages the application of the drugs in accord with the safety data set.

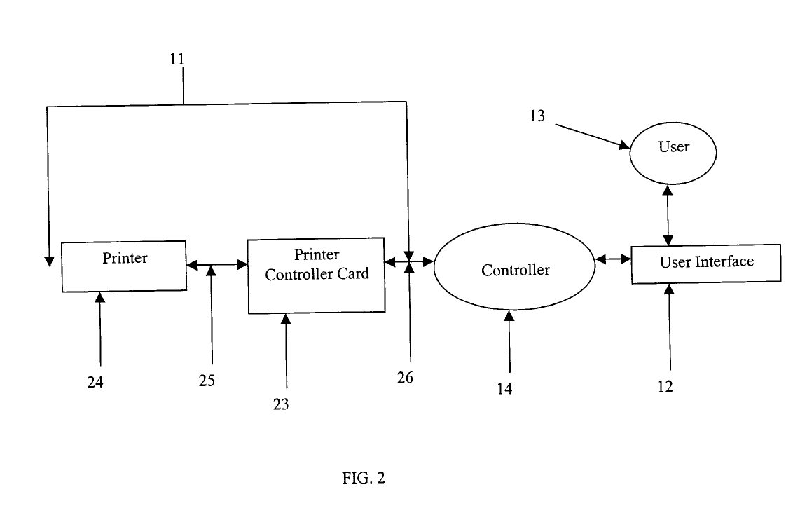

FIG. 2 illustrates a flow chart depicting one embodiment of printer system 11, in cooperation with controller 14 and user interface 12, where user interface 12 is operated by user 13. User 13 inputs data into user interface 12 via hard buttons, soft buttons, touch sensitive buttons, a voice recognition system, or by other suitable means of data entry such as, among other things, bar code readers. User interface 12 transfers the user-entered data to controller 14 where the information is gathered into a program designed in a language such as, for example, C or C++, functioning in an operating system such as, for example, QNX. Commands are sent from controller 14 to printer controller card 23 via port 26. Port 26 may be a parallel port, a serial port, USB, SCSI, FireWire, flexible port or other suitable means of transferring data. Printer controller card 23 is, in one embodiment, a CHARTKARD, manufactured by Parallel Systems Corporation, however printer controller card 23 may be any interface circuit suitable for transferring data from controller 14 to printer 24. A further embodiment of the present invention comprises printer system 11 having programming in place of the presence of a printer controller board. Printer controller card 23 functions to convert data received from controller 14 into commands transmittable to printer 23. Printer controller card 23 may be connected to printer 24 via port 25 which may be a parallel port, a serial port, a flexible port, a wireless port or by other suitable means of transferring data. Printer 24 is, in one embodiment of the present invention, a thermal micro printer, made by Seiko, however the present invention also contemplates the use of other printers suitable for use with printer system 11. A further embodiment of the present invention comprises a detachable printer (not shown), where the detachable printer may be affixed to sedation and analgesia system 22, or placed in any desirable position adjacent sedation and analgesia system 22. An even further embodiment of the present invention comprises a remote printer (not shown), where the remote printer may be adapted to receive wireless commands in the form of transmission waves from sedation and analgesia system 22, where the remote printer is adapted to function without direct connection to sedation and analgesia system 22.

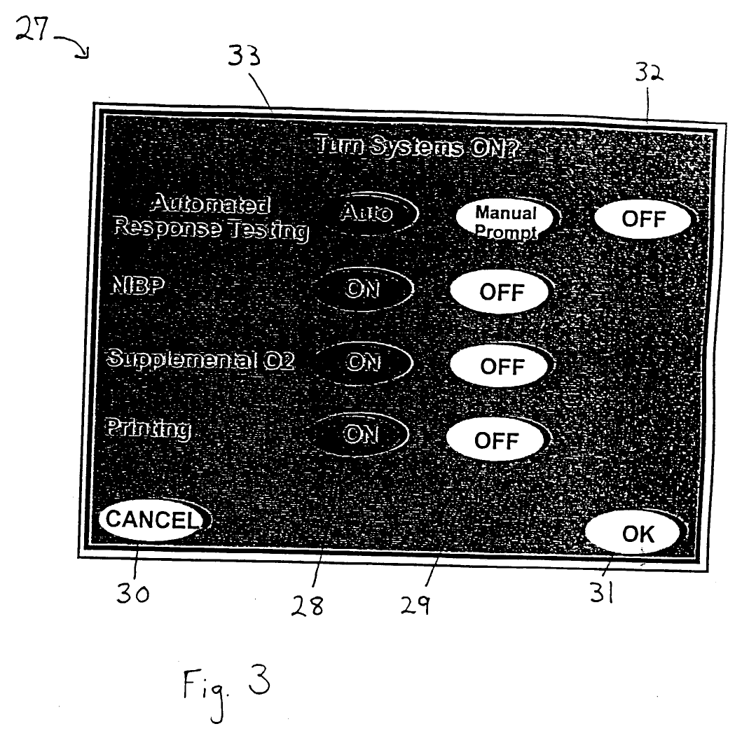

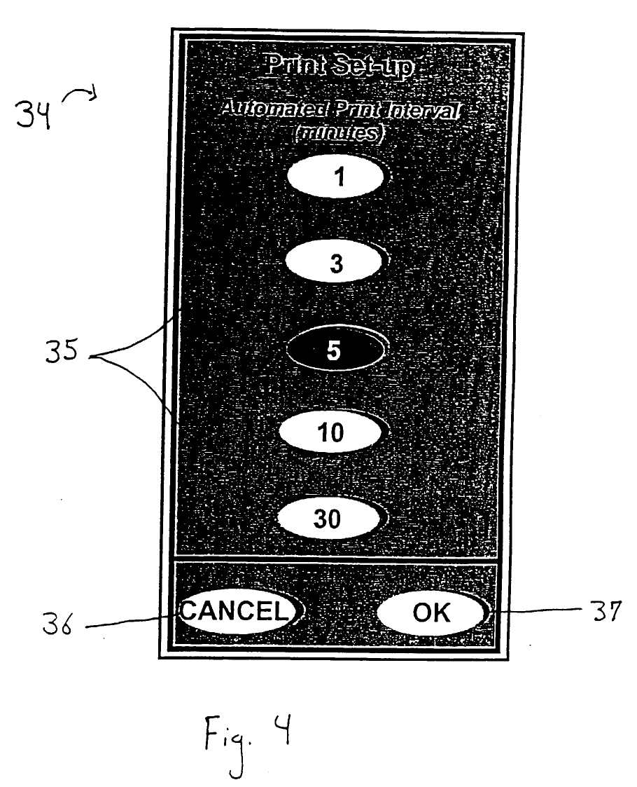

FIG. 3 illustrates one embodiment of an interface prompt 27 of user interface 12 having on button 28 to activate printer 24, off button 29 to deactivate printer 24, cancel button 30 to return to the main user interface prompt (not shown), off button 32 to shut down one or more sub-systems of sedation and analgesia system 22, auto button 33 to automatically commence printing in a default mode, and OK button 31 to prompt further information regarding printing. FIG. 3 shows these buttons as related to an Automated Responsiveness Test (ART) sub-system, but the buttons may be related to corresponding controls of other sub-systems of sedation and analgesia system 22. As illustrated, interface prompt 27 may be used to turn on or off other features of sedation and analgesia system 22 such as, for example, NIBP or supplemental oxygen. The buttons 28, 29, 30, 31, 32, 33 may be soft buttons, hard buttons, touch sensitive, responsive to verbal commands, or may be activated in other ways commonly known in the art. The presence of the on button 28 and off button 29 give the clinician the option of activating or deactivating the printer 24 at any time during the medical procedure, or the option of disabling the printer 24 for the duration of the procedure. This option is consistent with the “clinician knows best” philosophy giving the user 13 ultimate control as to what features of sedation and analgesia system 22 are necessary for a given procedure. In one embodiment of the present invention, activating OK button 31 prompts second interface prompt 34 illustrated in FIG. 4.

FIG. 4 illustrates one embodiment of second interface prompt 34 consistent with the present invention having interval buttons 35, cancel button 36, and OK button 37. Buttons 35, 36, 37 may be soft buttons, hard buttons, touch sensitive, responsive to verbal commands, or may be activated in other ways commonly known in the art. Interval buttons 35 function to print readings based on the average information retained in the sedation and analgesia system 22 memory over the period specified. For example, the selection of interval button 35 denoting a five minute time period will function to print out information desired by the clinician such as, for example, information used in meeting JCAHO standards, as an average of the variables requested by the clinician over the previous five minute period. Averaging the data received from the patient interface 17 (FIG. 1) over a set period of time allows the clinician to record data necessary to meet JCAHO standards while at the same time diminishing the impact of artifacts due to monitoring errors unrelated to the patient's true condition. Interval buttons 35 may offer the clinician a range of time periods from which to choose and may be changed at any time during the medical procedure depending on the needs of the particular application. A further embodiment of the present invention comprises the use of an LED screen (not shown) into which a time recording interval may be input via a keyboard, up/down selector, or by other suitable means. Second interface prompt 34 further comprises OK button 37 to accept the activated interval button 35, and a cancel button 36 to return to the previous prompt or main interface prompt.

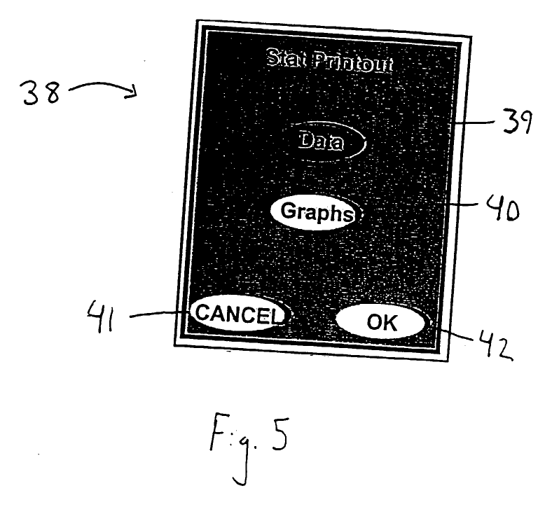

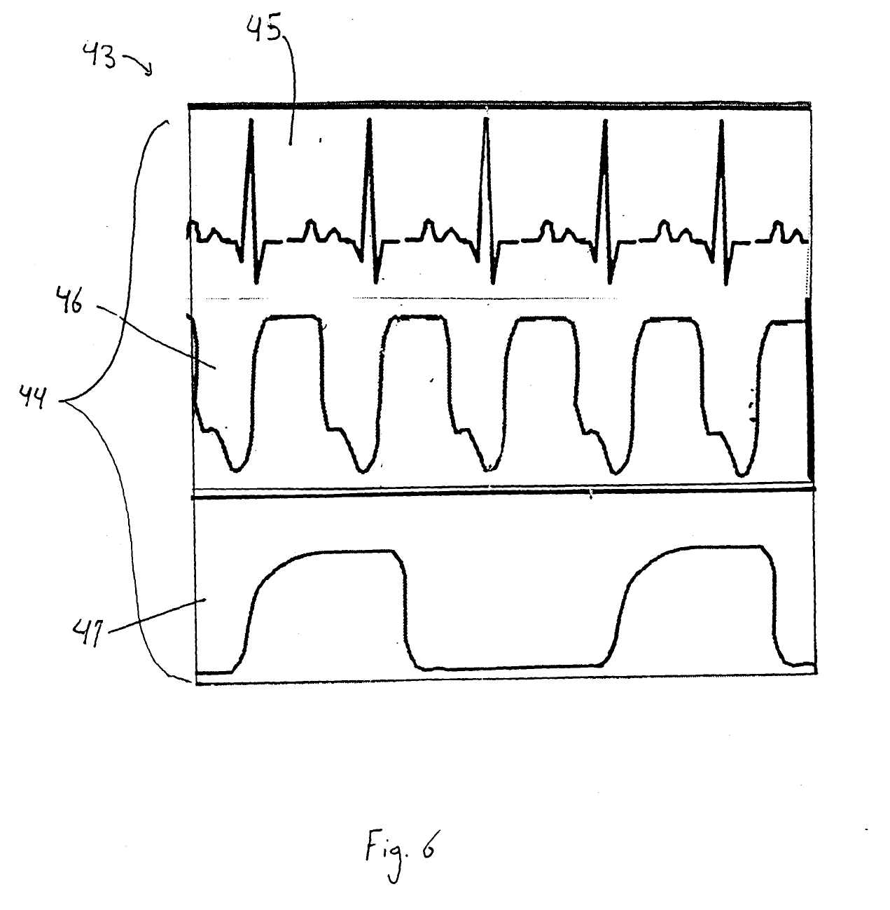

FIG. 5 illustrates a STAT prompt 38 having data button 39, graphs button 40, cancel button 41, and OK button 42. Buttons 39, 40, 41, 42 may be soft buttons, hard buttons, touch sensitive, responsive to verbal commands, or may be activated in other ways commonly known in the art. STAT prompt 38 may be selectable on the main user interface (not shown) at all times, may be activated by initiating a STAT command on another prompt, or may follow further printer prompts such as, for example, second interface prompt 34. In one embodiment of the present invention, STAT prompt 38 functions to provide a clinician with the ability to print out a 15 second (or some other suitable time) medical history depicting data received from the patient interface 17 in graph form if graphs button 40 is selected and in data form if data button 39 is selected. STAT prompt 38 provides the clinician, nurse, or other operator with the ability to print data containing anomalies that may require further examination. The STAT prompt 38 system used in cooperation with second interface prompt 34 allows for JCAHO data to be recorded that minimizes the impact of artifacts, yet at the same time provides the clinician ultimate control in printing out the aforementioned artifacts to determine if they have any relevance to the medical procedure at hand. Details of STAT graph printout 43 (FIG. 6) will be further discussed below. The present invention further comprises STAT prompt 38 having a variable time function, where the clinician may select a specific amount of time elapsed to print such as, for example, the last 10 seconds, 5 seconds, or any other desirable time period. A further embodiment comprises the ability to print the STAT printout 43 during the entire procedure depending on the needs of the clinician.

FIG. 6 illustrates one embodiment of STAT graph printout 43 in accordance with the present invention in the form of graphs 44, (in situations where graphs button 40 was pressed on STAT prompt 38) where the graphs 44 display the recent history of critical patient parameters. FIG. 6 illustrates graphs 44 comprising ECG graph 45, pulse oximetry graph 46, and carbon dioxide analysis graph 47, where graphs 44 illustrate real time data in one-to-one relationship with one another in order to provide a fast and efficient means of comparing data from one graph 44 with information from another. Comparative data gives the clinician the opportunity to determine whether an anomaly may be significant due to its relation to a number of other measurements of physical parameters. This system provides the operator with “orthogonal redundancy”, or a system of multiple checks and balances related to a single measured parameter in order to evaluate information in the context of interrelated data. Further embodiments of STAT printout 43 comprise the inclusion of data numbers from which the graph is constructed, graphs relating to parameters such as, for example, drug effect site concentration and/or blood pressure, time axes, concentration axes, input physician orders, and/or other data beneficial in characterizing a medical event. A further embodiment of the present invention comprises the exclusion of one or more graphs 44 illustrated in FIG. 6.

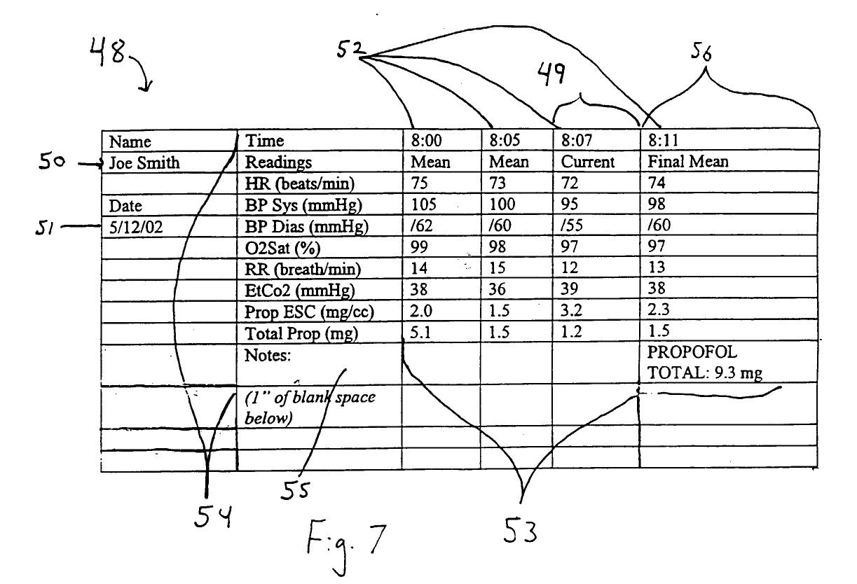

FIG. 7 illustrates one embodiment of interval data (numerical) printout 48 in accordance with the present invention having STAT data printout 49. In one embodiment of the present invention interval data printout 48 is initiated by interface prompt 27 and second interface prompt 34 and functions to print data related to JCAHO standards such as, for example, patient name input 50, date input 51, time readings 52, parameter readings 53, data headings 54, and notes input 55. As illustrated in FIG. 7, data related to selected parameters may be printed every five minutes as seen at 8:00 and 8:05 in order to record data that averages data recorded over the print, for example, five minute interval. Averaging this data minimizes the significance of artifacts, as previously discussed, while complying with JCAHO standards. STAT data printout 49 may be activated by the clinician at any time during the procedure and provides a “Current” reading of desired parameters over a given time interval that may be displayed as an average for a shortened period such as, for example, 15 seconds, or may be a real time spot check printout. This data may be used independent of, or in cooperation with, the STAT graph printout 43 in order to characterize data. In one embodiment of the present invention final data printout 56 provides the clinician with an overall mean for data acquired during the procedure. Time readings 52 may be established by the operator via second interface prompt 34 and inputting the desired time interval. Parameter readings 53 and data headings 54 may be entered or removed by the clinician in order to include or exclude data relevant or irrelevant to the medical application, respectively. Notes input 55 allows for the operator to input data relevant to the procedure that may not be captured by the pre-selected data parameters. Data may be entered such as, for example, drug administration, patient response data from, for example, the automated responsiveness test disclosed in commonly assigned and co-pending U.S. patent application Ser. No. 10/329,763 filed Dec. 27, 2002, or factors helpful in meeting JCAHO standards. It will be obvious to one of ordinary skill in the art that any parameter relevant to a medical procedure may be incorporated into interval data printout 48 and that any such feature may be included or omitted from the interval data printout 48 at the discretion of the clinician.

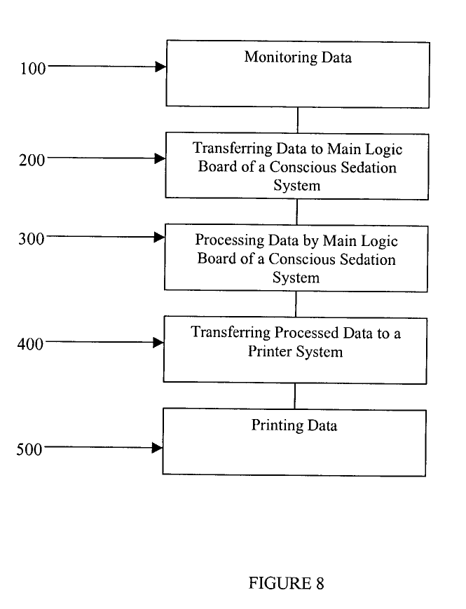

FIG. 8 illustrates a flow chart depicting a method of printing data monitored by a sedation and analgesia system comprising the steps of acquiring data from physiological monitors and sub-systems 100, transferring data to controller 14 of a sedation and analgesia system 200, processing data by controller 14 of a sedation and analgesia system 300, transferring processed data to a printer system 400, and printing data 500. Acquiring data from physiological monitors and sub-systems step 100 comprises acquiring data from patient interface 17 (FIG. 1), power supply 16, peripherals 15, user interface 12, external communications 10, printer system 11, drug delivery 19, or other features connectable to software controlled controller 14.

Transferring data to controller 14 of a sedation and analgesia system step 200 comprises the transfer of data to controller 14 of sedation and analgesia system 22 via parallel port, serial port, USB. SCSI, Firewire, A/D converter, flexible port, wireless output, or by other suitable means of transferring data.

Processing data by controller 14 of a sedation and analgesia system step 300 comprises the processing of data by software controlled controller 14 of a sedation and analgesia system 22 by utilizing programs inherent or incorporated into software controlled controller 14 desirable for use with a particular medical application such as, for example, printer specifications, data heading 54, and/or time readings 52.

Transferring processed data to a printer system step 400 comprises transferring data to printer system 11, where data may be transferred via parallel port, serial port, USB port, Firewire, SCSI, flexible port, or by other suitable transference means.

Printing data step 500 comprises printing a hard copy of data from a printer 24, where the printout from printer 24 may be interval data printout 48, STAT data printout 49, STAT graph printout 43, or other printout desirable for a medical procedure.

1. A sedation and analgesia system comprising:
a patient health monitor device adapted so as to be coupled to a patient and generate a signal reflecting at least one physiological condition of the patient;
a drug delivery controller supplying one or more drugs to the patient;
a memory device storing a safety data set reflecting safe and undesirable parameters of at least one monitored patient physiological condition;
an electronic controller interconnected between the patient health monitor, the drug delivery controller and the memory device storing the safety data set; wherein said electronic controller receives said signal and in response manages the application of the drugs in accord with the safety data set; and
a printing system for outputting one or more of system state information and patient state information, wherein the printing system is interconnected with the electronic controller such that the electronic controller manages the output of the printing system.

2. The sedation and analgesia system according to claim 1, wherein the printing system is remote from the electronic controller.

3. The sedation and analgesia system according to claim 1 further comprising an interface for controlling one or more functions and one or more settings of the printer system.

4. The sedation and analgesia system according to claim 1, wherein the printing system outputs a first set of information at a first time and a second set of information at a second time, and wherein there is a pre-determined interval of time between the first time and the second time.

5. The sedation and analgesia system according to claim 4, wherein the first and second sets of information comprise statistical data, the statistical data being the average of data that is collected over the pre-determined interval.

6. The sedation and analgesia system according to claim 4, further comprising an interface for receiving a value for the pre-determined interval, the interface being interconnected with the electronic controller.

7. The sedation and analgesia system according to claim 1, wherein the one or more of system state information and patient state information comprises one or more graphs.

8. The sedation and analgesia system according to claim 7, wherein the one or more graphs comprise one or more of ECG data, pulse oximetry data, carbon dioxide analysis data, and drug effect site concentration data.

9. The sedation and analgesia system according to claim 1, wherein the patient state information is derived from the signal reflecting at least one physiological condition of the patient.

10. The sedation and analgesia system according to claim 1, wherein the patient state information comprises one or more of ECG data, pulse oximetry data, carbon dioxide analysis data, and drug effect site concentration data.

11. The sedation and analgesia system according to claim 1 further comprising a memory device storing the most recently overwritten data from an associated display device such as a video display where (sub) for example the interval is 15 seconds.

12. A method of operating a sedation and analgesia system comprising the steps of:
connecting to a patient a drug delivery device having a drug delivery controller supplying one or more drugs, said drug delivery controller being coupled to an electronic controller which controls the delivery of the drugs to the patient;
attaching at least one patient health monitor device to a patient, which health monitor device generates a value reflecting at least one physiological condition of a patient and is coupled to said electronic controller;
accessing a memory device which stores a safety data set reflecting parameters of at least one patient physiological condition;
delivering the drugs to the patient in accord with the safety data set; and
printing one or more of system state information and patient state information.

13. The method of operating a sedation and analgesia system according to claim 12, wherein the step of printing comprises the steps of printing a first set of information at a first time, and printing a second set of information at a second time, wherein there is a pre-determined interval of time between the first time and the second time.

14. The method of operating a sedation and analgesia system according to claim 13, wherein the first and second sets of information comprise statistical data, wherein the step of printing further comprises the step of averaging the data of the first and second sets of information, the average being over the predetermined interval of time, and wherein the step of printing further comprises the step of printing the statistical average of the data.

15. The method of operating a sedation and analgesia system according to claim 13 further comprising the step of inputting the pre-determined interval of time with a user interface.

16. The method of operating a sedation and analgesia system according to claim 12, wherein the one or more of system state information and patient state information comprises one or more graphs.

17. The method of operating a sedation and analgesia system according to claim 16, wherein the one or more of system state information and patient state information comprises two or more graphs, and wherein the method further comprises the step of comparing the two or more graphs against each other.

18. The method of operating a sedation and analgesia system according to claim 12, wherein the step of printing is performed immediately upon a request for said step.

19. The method of operating a sedation and analgesia system according to claim 18, wherein the one or more of system state information and patient state information printed comprises graphical data.

20. The method of operating a sedation and analgesia system according to claim 18, wherein the one or more of system state information and patient state information printed comprises numerical data.

21. The method of operating a sedation and analgesia system according to claim 12, wherein the patient state information that is printed is derived from at least one physiological condition of a patient.

22. The method of operating a sedation and analgesia system according to claim 12, wherein the patient state information that is printed is derived from one or more of ECG data, pulse oximetry data, carbon dioxide analysis data, and drug effect site concentration data.

23. The method of operating a sedation and analgesia system according to claim 12, further comprising the step of automatically entering regulatory compliance data into fields of a user interface.
